(19) United States
(12) Patent Application Publication
(10) Pub. No.: US 2024/0341732 A1
(43) Pub. Date: Oct. 17, 2024

(54) INTRALUMINAL MEDICAL SYSTEM WITH OVERLOADED CONNECTORS

(71) Applicant: PHILIPS IMAGE GUIDED THERAPY CORPORATION, SAN DIEGO, CA (US)

(72) Inventors: Joseph James HOFFMAN, SACRAMENTO, CA (US); Cesar PEREZ, ROSEVILLE, CA (US); Sherwood KANTOR, SACRAMENTO, CA (US)

(21) Appl. No.: 18/751,414

(22) Filed: Jun. 24, 2024

Related U.S. Application Data
(63) Continuation of application No. 16/756,406, filed on Apr. 15, 2020, now abandoned.
(60) Provisional application No. 62/574,835, filed on Oct. 20, 2017.

(51) Int. Cl. A61B 8/00; A61B 8/12; G16H 30/20; G16H 40/67

(52) U.S. Cl. CPC A61B 8/56; A61B 8/12; A61B 8/4411; A61B 8/585; G16H 30/20; G16H 40/67; A61B 2562/0247

(57) ABSTRACT

Medical devices, systems and methods are provided. In one embodiments, an intraluminal medical system includes a patient interface module (PIM) configured to selectively communicate with a first intraluminal device or a second intraluminal device different than the first intraluminal device. The first and second intraluminal devices are configured to obtain medical data associated with a body lumen of a patient while positioned within the body lumen. The PIM includes a first connector having a first plurality of pins respectively carrying a plurality of signals. The first intraluminal device includes a second connector configured to engage the first connector and the second intraluminal device includes a third connector configured to engage the first connector. The plurality of signals respectively carried by the first plurality of pins is configured to selectively allow electrical communication between the PIM and the first intraluminal device when the second connector engages the first connector, and between the PIM and the second intraluminal device when the third connector engages the first connector.

### CROSS-REFERENCE TO PRIOR APPLICATIONS

This application is a continuation of U.S. application Ser. No. 16/756,406, filed on Apr. 15, 2020, which is the U.S. National Phase application under 35 U.S.C. § 371 of International Application No. PCT/EP2018/078791, filed on Oct. 19, 2018, which claims the benefit of U.S. Provisional Patent Application No. 62/574,835, filed on Oct. 20, 2017. These applications are hereby incorporated by reference herein.

### TECHNICAL FIELD

The present disclosure relates generally to patient interface modules (PIM) of intraluminal medical systems and, in particular, to PIMs that are compatible with the overloaded connectors. For example, the PIM of the present disclosure can electrically communicate with one of several different types of intraluminal devices and configure the intraluminal medical system based on the type of intraluminal device in communication with the PIM.

### BACKGROUND

Catheters are widely used as diagnostic tools for assessing a diseased vessel, such as an artery, within the human body to determine the need for treatment, to guide the intervention, and/or to assess its effectiveness. Catheter come equipped with different types of sensors, such as ultrasound transducers, optical sensors, flow rate sensors, pressure sensors and photoacoustic sensors. Conventionally, catheters with different sensor types can have different connectors that go with different patient interface modules (PIM). Ultrasound catheters operating at different center frequencies traditionally share connectors of identical dimensions. However, due to the difference in the number of pins and the power outputs, ultrasound catheters operating at different center frequencies are connected to different PIMs. As a result, using different types of catheters in a catheter lab involves use of multiplicity of connectors and multiplicity of PIMs, complicating the workflow, increasing the cost, and increasing the number of failure points.

### SUMMARY

Embodiments of the present disclosure provide an intraluminal medical system that includes a patient interface module (PIM) that can selectively communicate with a different intraluminal devices (e.g., catheters, guidewires, etc.) with different types of sensors. The PIM connects to the different intraluminal devices using a single PIM connector. The PIM connectors include multiple pins that carry different electrical signals (e.g., ground, power, signal, data, etc.). The multiple pins are arranged such that the PIM can communicate with the different intraluminal devices when the different intraluminal devices are respectively connected to the single PIM connector.

In one embodiments, an intraluminal medical system includes a patient interface module (PIM) configured to selectively communicate with a first intraluminal device or a second intraluminal device different than the first intraluminal device. The first and second intraluminal devices are configured to obtain medical data associated with a body lumen of a patient while positioned within the body lumen. The PIM includes a first connector having a first plurality of pins respectively carrying a plurality of signals. The first intraluminal device includes a second connector configured to engage the first connector and the second intraluminal device includes a third connector configured to engage the first connector. The plurality of signals respectively carried by the first plurality of pins is configured to selectively allow electrical communication between the PIM and the first intraluminal device when the second connector engages the first connector, and between the PIM and the second intraluminal device when the third connector engages the first connector.

In some embodiments, the intraluminal medical system further includes the first intraluminal device and the second intraluminal device. In some embodiments, the first intraluminal device includes a first type of ultrasound transducers and the second intraluminal device includes a second type of ultrasound transducers. The first type of ultrasound transducers is different from the second type of ultrasound transducers. In some embodiments, the first intraluminal device includes ultrasound transducers with a first center frequency, and the second intraluminal device includes ultrasound transducers with a second center frequency different from the first center frequency. In some implementations, the second connector includes a first pin configuration and the third connector includes a second pin configuration different from the first pin configuration. In some implementations, when the second connector engages the first connector, a first subset of the first plurality of pins are open; and when the third connector engages the first connector, a second subset of the first plurality of pins are open. The first subset is different from the second subset. In some embodiments, when the second connector engages the first connector, the plurality of signals experiences a first change; and when the third connector engages the first connector, the plurality of signals experience a second change. In these embodiments, the PIM is operable to detect the first change, thereby identifying the first intraluminal device. In addition, in these embodiments, the PIM is operable to detect the second change, thereby identifying the second intraluminal device.

In some embodiments, when first intraluminal device is identified, the PIM is operable to configure itself and the intraluminal medical system based on attributes of the first intraluminal device. In some embodiments, when second intraluminal device is identified, the PIM is operable to configure itself and the intraluminal medical system based on attributes of the second intraluminal device. In some implementations, the second connector includes a second plurality of pins and the third connector includes a third plurality of pins. At least one of the second plurality of pins is connected to a first electrically erasable programmable read-only memory (EEPROM) storing first data. At least one of the third plurality of pins is connected to a second EEPROM storing second data different from the first data. In these implementations, the PIM is operable to read the first data, thereby identifying the first intraluminal device, and the PIM is further operable to read the second data, thereby identifying the second intraluminal device. In some embodiments, when first intraluminal device is identified, the PIM is operable to configure itself and the intraluminal medical system based on attributes of the first intraluminal device. In some embodiments, when second intraluminal device is identified, wherein the PIM is operable to configure itself and the intraluminal medical system based on attributes of the second intraluminal device.

In another embodiment, a method for selectively establishing communication between a patient interface module (PIM) and different intraluminal devices is provided. The method includes identifying, by use of the PIM, a first intraluminal device by detecting a first change in signals carried by a first connector of the PIM, when a second connector of the first intraluminal device engages the first connector; and identifying, by use of the PIM, a second intraluminal device by detecting a second change in signals carried by the first connector of the PIM, when a third connector of the second intraluminal device engages the first connector. In this embodiments, the first and second intraluminal devices are configured to obtain medical data associated with a body lumen of a patient while positioned within the body lumen. The second connector includes a first pin configuration and the third connector includes a second pin configuration different from the first pin configuration. In some implementations, the method further includes configuring the PIM based on attributes of the first intraluminal device when first intraluminal device is identified and configuring PIM based on attributes of the second intraluminal device when second intraluminal device is identified. In some implementations, the PIM is in communication with a console. In some embodiments, the method further includes configuring the PIM and the console based on attributes of the first intraluminal device when first intraluminal device is identified, and configuring the PIM and the console based on attributes of the second intraluminal device when second intraluminal device is identified. In some embodiments, the first and second changes include a change in impedance, a change in current output, or a change due to reading of data stored on a memory of the first or second intraluminal device.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic schematic view of a prior-art intraluminal medical system that includes multiplicity of PIMs.

FIG. 2 is a diagrammatic schematic view of an intraluminal medical system with a single PIM, according to aspects of the present disclosure.

FIGS. 3A and 3B are schematic diagrams of two connectors configured to engage a connector on a PIM, according to aspects of the present disclosure.

FIG. 4 is an illustrative table of exemplary connector pin configurations of the connectors in FIGS. 3A and 3B, according to aspects of the present disclosure.

FIGS. 5A and 5B are schematic diagrams of two connectors configured to engage a connector on a PIM, according to aspects of the present disclosure.

FIG. 6 is an illustrative table of exemplary connector pin configurations of the connectors in FIGS. 5A and 5B, according to aspects of the present disclosure.

FIG. 7 is an illustrative table of exemplary connector pin configurations of the connectors in FIGS. 5A and 5B, according to aspects of the present disclosure.

FIGS. 8A and 8B are schematic diagrams of two connectors configured to engage a connector on a PIM, according to aspects of the present disclosure.

FIG. 9 is an illustrative table of exemplary connector pin configurations of the connectors in FIGS. 8A and 8B, according to aspects of the present disclosure.

FIG. 10 is a schematic diagram of a connector configured to engage a connector on a PIM, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

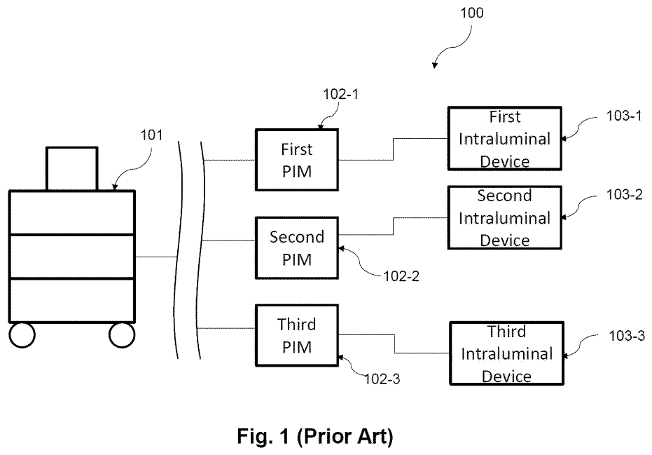

FIG. 1 is a diagrammatic schematic view of a prior-art intraluminal medical system 100. The prior-art intraluminal medical system 100 includes a cart-based console 101 that can be connected to different intraluminal devices with different sensor types, such as a first intraluminal device 103-1, a second intraluminal device 103-2 and a third intraluminal device 103-3. For example, the first intraluminal device 103-1 can be an intravascular ultrasound (IVUS) catheter with ultrasound transducers operating at 10 MHz. The second intraluminal device 103-2 can be an IVUS catheter with ultrasound transducers operating at 20 MHz. The third intraluminal device 103-3 can be a pressure catheter with pressure sensors. In some situations, the first, second and third intraluminal devices 103-1, 103-2 and 103-3 can have connectors of identical dimensions. In some other situations, they can have entirely different connectors. Conventionally, because the signal and power transmitted to and from the first, second and third intraluminal devices 103-1, 103-2, and 103-3 vary, each of them is paired to a sensor-type-specific PIM. As shown in FIG. 1, the first intraluminal device 103-1 is paired to a first PIM 102-1, the second intraluminal device 103-2 is paired to a second PIM 102-2, and the third intraluminal device 103-3 is paired to a third PIM 102-3. Each of the PIMs is connected to the console 101. Consequently, in order to perform catheterization with the first, second and third intraluminal devices 103-1, 103-2 and 103-3, the clinician has to set up different PIMs and make sure that each of the intraluminal devices is connected to the corresponding sensor-type specific PIM. The requirement of multiple PIMs increases the cost of equipment, takes up valuable space in the catheter lab, reduces the reliability of catheterization, increases the number of failure points, and increase chance of user errors.

Referring now to FIG. 2, shown therein is a diagrammatic schematic view of an intraluminal medical system 200 according to aspects of the present disclosure. The intraluminal medical system 200 includes a PIM 202 that can establish electrical communication with a plurality of intraluminal devices with different types of sensors. In embodiments presented by FIG. 2, the PIM 202 is connected to a console 201 by a cable and also includes a connector 204. The connector 204 may be referred to herein from time to time as PIM connector 204. The PIM connector 204 can be used to establish communication between the PIM 202 and a plurality of intraluminal devices with different types of sensors, such a first intraluminal device 203-1, a second intraluminal device 203-2, and a N^{th} intraluminal device 203-N. The first, second and N^{th} intraluminal devices 203-1, 203-2 and 203-N are configured to be inserted into a body lumen of a patient to obtain physiology data of the body lumen. The first, second and N^{th} intraluminal devices 203-1, 203-2 and 203-N may be IVUS devices operating at different center frequencies or IVUS catheter with different ultrasound transducers. Generally, an IVUS device can include a single ultrasound transducer or multiple ultrasound transducer elements, such as in an ultrasound transducer array.

The intraluminal devices 203-1, 203-2 and 203-N include a flexible elongate member sized and shaped, structurally arranged, and/or otherwise configured to be positioned within anatomy of a patient. One or more sensing components can be positioned at the distal portion of the flexible elongate member. Generally, intraluminal devices 203-1, 203-2 and 203-N can be guidewires, catheters, guide catheters, and/or combinations thereof. One or more of the intraluminal devices 203-1, 203-2 and 203-N can be a rotational IVUS imaging device including a rotating drive cable that rotates one or more ultrasound transducers at the distal portion of the flexible elongate member. One or more of the intraluminal devices 203-1, 203-2 and 203-N can be a phased array IVUS imaging device including a circumferential/annular transducer array around a longitudinal axis. In other embodiments, the sensing component of the intraluminal devices 203-1, 203-2 and 203-N can be configured for imaging, such as near infrared (NIR) imaging, optical coherence tomography (OCT), intravascular photoacoustic (IVPA) imaging, transesophageal echocardiography (TEE), and intracardiac echocardiography (ICE). In some embodiments, one or more of the intraluminal devices 203-1, 203-2 and 203-N can include any suitable sensing component, including a pressure sensor, a flow sensor, a temperature sensor, an optical fiber, a reflector, a mirror, a prism, an ablation element, a radio frequency (RF) electrode, a conductor, and/or combinations thereof.

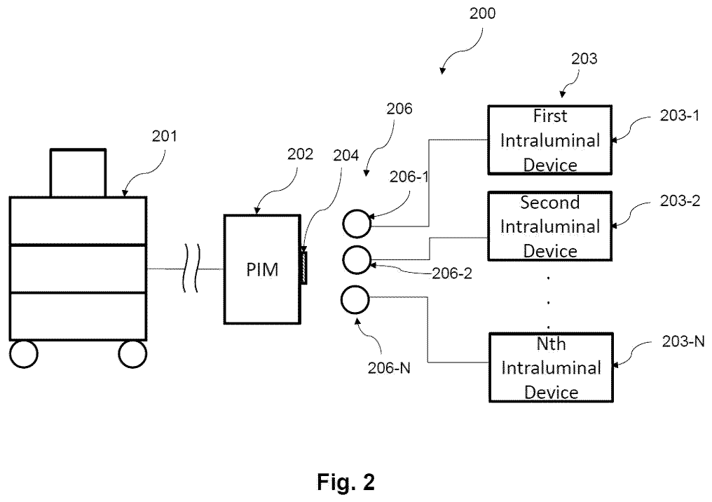

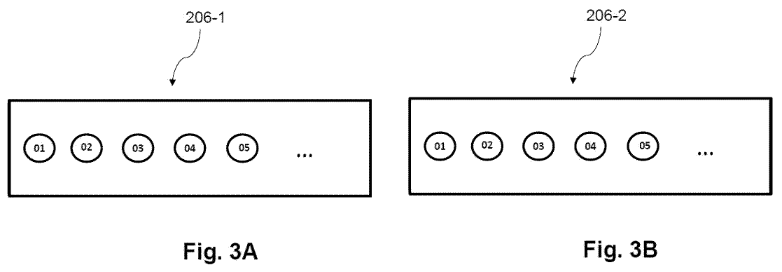

While IVUS catheters operating at different center frequencies and IVUS catheter with different ultrasound transducers all include ultrasound transducers, these IVUS catheters are considered different of intraluminal devices or having different types of ultrasound transducers. As used herein, N stands for an integer, representing the number of different types of intraluminal devices that can be compatible with the PIM 202. The first intraluminal device 203-1 includes a first device connector 206-1, the second intraluminal device 203-2 includes a second device connector 206-2 and the N^{th} intraluminal device 203-N includes an N^{th} device connector 206-N. For the case of reference, the first, second and N^{th} device connectors may be referred to individually as device connector 206-1, device connector 206-2 and device connector 206-N or together as device connectors 206. Each of device connectors 206 is compatible with the PIM connector 204 and can engage PIM connector 204 to establish electrical communication between the respective intraluminal device and the PIM 202. In some embodiments, the device connectors 206-1, 206-2 and 206-N can include protrusions, recessions, or other structural or mechanical features that can match corresponding features on the PIM connector 204 for secured and reliable connection. As will be further described below in conjunction with FIGS. 3A to 10, while device connectors 206 are identical in physical dimensions, they have different configurations in various embodiments of the present disclosure. These different configurations allow the PIM 202 to identify the sensor type of the intraluminal device that is currently connected to the PIM 202. Once the sensor type of the intraluminal device is identified, the PIM 202 can, in some implementations, configure itself and the console 201 based on the attributes of the identified sensor type.

The body lumen, as used herein, can be a vessel, such as a blood vessel. In various embodiments, the body lumen is an artery or a vein of a patient's vascular system, including cardiac vasculature, peripheral vasculature, neural vasculature, renal vasculature, and/or any other suitable anatomy/lumen inside the body. The body lumen can be tortuous in some instances. For example, the first, second and N^{th} intraluminal devices 203-1, 203-2 and 203-N may be used to examine any number of anatomical locations and tissue types, including without limitation, organs including the liver, heart, kidneys, gall bladder, pancreas, lungs, esophagus; ducts; intestines; nervous system structures including the brain, dural sac, spinal cord and peripheral nerves; the urinary tract; as well as valves within the blood, chambers or other parts of the heart, and/or other systems of the body. In addition to natural structures, the first, second and N^{th} intraluminal devices 203-1, 203-2 and 203-N may be used to examine man-made structures such as, but without limitation, heart valves, stents, shunts, filters and other devices.

The console 201 can include a processing circuit, such as one or more processors in communication with memory. The console 201 can receive, process, and generate a graphical representation of the intraluminal data obtained by the intraluminal devices 203-1, 203-2, 203-N. The console 201 can transmit the graphical representation of the intraluminal data to a display for display to a user. The console 201 can include a user input device to allow a user to control operation of the intraluminal devices 203-1, 203-2, 203-N. The console 201 can transmit control signals to the intraluminal devices 203-1, 203-2, 203-N, e.g., based on received user input.

The intraluminal medical system 200 of the present disclosure provides several advantages over the conventional design. By using a single PIM to connect to different types of intraluminal devices via a single type of standardized connector pairs (i.e. a PIM connector and a compatible device connect being a pair), the workflow becomes simpler and more streamlined, the number of PIMs and connecting cables are reduced, equipment cost is lowered, procedural robustness and reliability are increased, and the number of failure points are reduced. As the PIM connector 204 can engage up to N device connectors 206 to selectively establish electrical communication between the PIM 202 and one of the intraluminal devices 203, the PIM connector 204 can be referred to as an overloaded connector with overloaded functions.

The PIM 202 can comprise a housing having any suitable shape. The PIM 202 can include a volume (e.g., a length, a width, a depth, a radius, etc.) within the housing configured to accommodate one or more components described herein. For example, the connector 204 can be mechanically coupled to the housing of the PIM 202. The PIM 202 can be sized and shaped, structurally arranged, and/or otherwise configured for handheld use in some embodiments.

In some embodiments, the intraluminal medical system 200 and/or the PIM 202 can include features similar to those described in U.S. Patent Application No. 62/574,455, titled “DIGITAL ROTATIONAL PATIENT INTERFACE MODULE,” filed Oct. 19, 2017, U.S. Patent Application No. 62/574,655, titled “WIRELESS DIGITAL PATIENT INTERFACE MODULE USING WIRELESS CHARGING,” filed Oct. 19, 2017, U.S. Patent Application No. 62/574,687, titled “INTRALUMINAL DEVICE REUSE PREVENTION WITH PATIENT INTERFACE MODULE AND ASSOCIATED DEVICES, SYSTEMS, AND METHODS,” filed Oct. 19, 2017, and U.S. Patent Application No. 62/574,610, titled “HANDHELD MEDICAL INTERFACE FOR INTRALUMINAL DEVICE AND ASSOCIATED DEVICES, SYSTEMS, AND METHODS,” filed Oct. 19, 2017, each of which is incorporated by reference in its entirety.

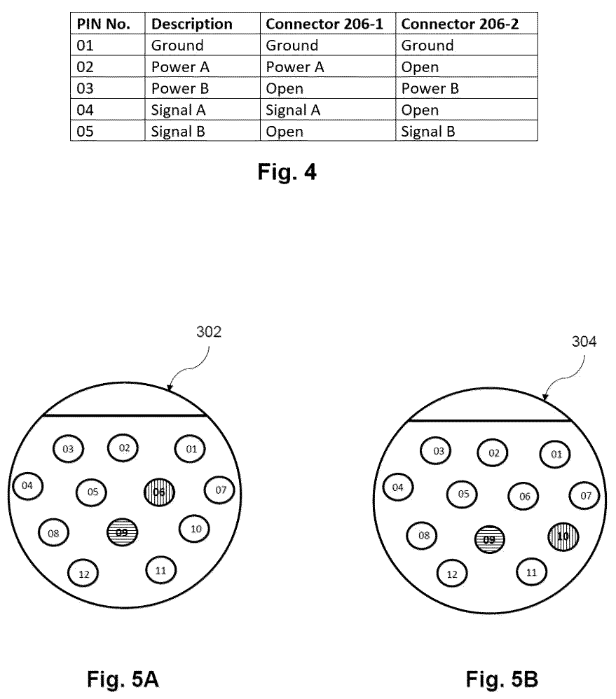

FIGS. 3A and 3B are schematic diagrams of the first device connector 206-1 and second device connector 206-2. In some embodiments, both of the connectors 206-1 and 206-2 include at least five pins-01, 02, 03, 04 and 05. In some implementations, the connectors 206-1 and 206-2 have the same number of pins. In some other implementations, the connector 206-1 includes more pins than the connector 206-2. In some embodiments, while the connector 206-1 and 206-2 share the five common pins as shown, the five pins in the connector 206-1 are configured differently from those in the connector 206-2. Referring now to FIG. 4, shown therein is an illustrative table of exemplary connector pin configurations of the connectors 206-1 and 206-2. In this exemplary table, the “PIN No” and “Description” columns describe the pin numbers and function of the PIM connector 204, with which the connectors 206-1 and 206-2 are compatible. In other words, in this example, pin 01 of the PIM 202 is grounded, pin 02 of the PIM 202 is a power pin and is referred to as “Power A,” pin 03 of the PIM 202 is another power pin and is referred to as “Power B,” pin 04 of the PIM 202 supplies a signal and is referred to as “Signal A,” and pin 05 of the PIM 202 supplies another signal and is referred to as “Signal B.” In this illustrative example, the connector 206-1 has corresponding pin 03 and pin 05 open, meaning that the connector 206-1 either has no corresponding pins 03 and 05 or its corresponding pins 03 and 05 are connected to an open circuit. The connector 206-2 has a different pin configuration where the corresponding pins 02 and 04 are open. In operation, the connector 206-1, being connected to the first intraluminal device 203-1 with a first sensor type, draws power from pin 02, but not from pin 03 and exhibits impedance at pin 04, but not at pin 05. The connector 206-2, being connected to the second intraluminal device 203-2 with a second sensor type, draws currently from pin 03, but not from pin 02, and exhibits impedance at pin 05, but not at pin 04. As result, the PIM 202 can identify the sensor types of the intraluminal device currently connected to itself based on pin-specific currently output, pin-specific impedance, and a combination of the two. In this example, just by presence of current draw and impedance, the exemplary five pin arrangement can allow the PIM 202 to identify, e.g., 9 types of sensors (when a connector can only be open in one of the power pins and one of the signal pins, but not both). If the current draw and impedance can be detectably different among different sensor types, the PIM 202 can identify a lot more types of sensors. Due to the pin configuration illustrated in FIG. 4, the first intraluminal device 203-1, by its connection to the device connector 206-1, makes use of a subset (pins 01, 02 and 04) of the available pins while leaving pins 03 and 05 open. The second intraluminal device 203-2, by its connection to the device connector 206-2, makes use of a different subset (pins 01, 03 and 05) of the available pins while leaving pins 02 and 04 open. Conversely, every subset of used pin corresponds to a subset of “open” or “open-circuit” pins. In some implementations, additional intraluminal device can be made to use pin subsets different from the aforementioned two subsets to achieve detectability by the PIM 202.

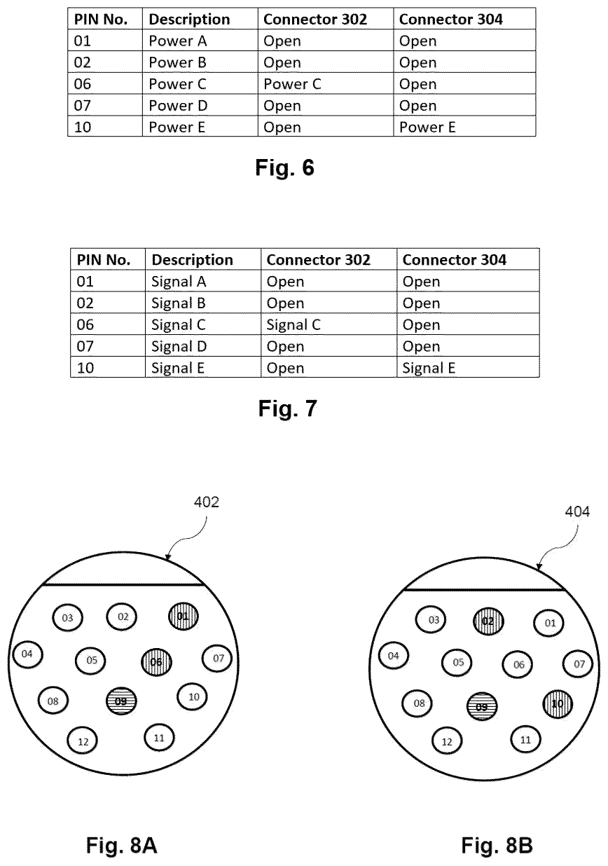

FIGS. 5A and 5B are schematic diagrams of a device connector 302 and a device connector 304. In some embodiments represented by FIGS. 5A and 5B, both the device connectors 302, 304 and the compatible PIM connector are 12-pin connectors. In some embodiments, the 12 pins in a 12-pin device connector may include a ground pin, a clock pin for synchronization, one or more amplifier pins, one or more trigger pins, one or more voltage/power pins, one or more pins for device identification, and one or more open pins. In some examples illustrated in the table in FIG. 6, pins 01, 02, 06, 07, and 10 of the PIM connector are all power pins-Power A, Power B, Power C, Power D and Power E. The device connector 302, being connected to an intraluminal device with a first type of sensor, draws power only at corresponding pin 06 while all the other corresponding pins 01, 02, 07 and 10 are left open-circuit. The device connector 304, being connected to an intraluminal device with a second type of sensor, draws power only at corresponding pin 10 while all of the other corresponding pins 01, 02, 06, and 07 are left open-circuit. By detecting the power draw at pin 06, the PIM, such as the PIM 202 in FIG. 2, can identify the first type of sensor when the device connector 302 engages the PIM connector. Additionally, by detecting the power draw at pin 10, the PIM, such as the PIM 202 in FIG. 2, can identify the second type of sensor when the device connector 304 engages the PIM connector.

FIG. 7 shows another illustrative table of exemplary connector pin configurations of the device connectors 302 and 304 in FIGS. 5A and 5B. In this example, pins 01, 02, 06, 07, and 10 of the PIM connector are all signal pins-Signal A, Signal B, Signal C, Signal D, and Signal E. The device connector 302, being connected to an intraluminal device with a first type of sensor, exhibits certain impedance at corresponding pin 06 while all the other corresponding pins 01, 02, 07 and 10 are left open-circuit. The device connector 304, being connected to an intraluminal device with a second type of sensor, exhibits certain impedance at corresponding pin 10 while all of the other corresponding pins 01, 02, 06, and 07 are left open-circuit. As the Signal C sees impedance at pin 06, the PIM can identify the first type of sensor when the device connector 302 engages the PIM connector. Additionally, as the Signal E sees impedance at pin 10, the PIM, such as the PIM 202 in FIG. 2, can identify the second type of sensor when the device connector 304 engages the PIM connector.

FIGS. 8A and 8B are schematic diagrams of a device connector 402 and a device connector 404. In some embodiments represented by FIGS. 8A and 8B, both the device connectors 402, 404 and the compatible PIM connector are 12-pin connectors. Similar to the device connectors 302 and 304 in FIGS. 5A and 5B, the 12 pins in a 12-pin device connector may include a ground pin, a clock pin for synchronization, one or more amplifier pins, one or more trigger pins, one or more voltage/power pins, one or more pins for device identification, and one or more open pins. In some examples illustrated in the table in FIG. 9, pins 01 and 02 of the PIM connector are power pins-Power A and Power B; and pins 06, 07 and 10 of the PIM connector are signal pins-Signal A, Signal B and Signal C. The device connector 402, being connected to an intraluminal device with a first type of sensor, draws power at corresponding pin 01 and exhibits certain impedance at corresponding pin 06 while all the other corresponding pins 02, 07 and 10 are left open-circuit. The device connector 404, being connected to an intraluminal device with a second type of sensor, draws power at corresponding pin 02 and exhibits certain impedance at corresponding pin 10, while all of the other corresponding pins 01, 06 and 07 are left open-circuit. By detecting the power draw at pin 01 and the impedance at pin 06, the PIM, such as the PIM 202 in FIG. 2, can identify the first type of sensor when the device connector 402 engages the PIM connector. Additionally, by detecting the power draw at pin 02 and the impedance at pin 10, the PIM, such as the PIM 202 in FIG. 2, can identify the second type of sensor when the device connector 404 engages the PIM connector.

As described above in conjunction with FIGS. 3A, 3B, 4, 5A, 5B, 6, 7, 8A, 8B, and 9, the PIM connector, such as the PIM connector 204, includes a plurality of pins. In the example illustrated in FIGS. 3A and 3B, the PIM connector there includes at least 5 pins. In the example illustrated in FIGS. 5A, 5B, 8A, and 8B, the PIM connector includes 12 pins. The plurality of pins of the PIM connector carries a plurality of signals. In the example illustrated in FIGS. 3A, 3B and 4, the plurality of pins of the PIM connector carries at least Signal A and Signal B. If Power A and Power B include voltage output and can be regarded as signals, the plurality of pins of the PIM connector carries additional signals associated with the Power A and Power B pins. In the example illustrated in FIGS. 5A, 5B, 6, 7, 8A, 8B, and 9, the plurality of pins of the PIM connector can carry five signals, five power outputs, or 2 power outputs and 3 signals, as the case may be. There, the plurality of pins of the PIM connector carries a plurality of signals as well. If each of the plurality of pins of the PIM connector carries some default level of signal, voltage or power output, engaging the PIM connector with a device connector connected to an intraluminal device can cause a change in the plurality of signals carried by the plurality of pins of the PIM connector. Such a change can be a change in impedance in one or more of the pins or a change in current output levels in one or more of the pins. By detecting the change in the plurality of signals carried by the plurality of pins of the PIM connector, the PIM can detect and identify a sensor type of an intraluminal device connected to the PIM.

In some embodiments, the PIM, such as the PIM 202 in FIG. 2, can enter into a “sleeping mode” when left idle or unconnected to any intraluminal device for a predetermine length of time. In the “sleeping mode,” the power consumption of PIM is maintained at a low level and the PIM does not transmit any control signals to the intraluminal device connected thereto. In some embodiments, when in the “sleeping mode,” the PIM can constantly or periodically output a sensing signal via all signal pins (such as Signal A, B, and C) of the PIM connector or a standby voltage at all power pins (such as Power A, B, and C) of the PIM connector. When any of the signal pins is not connected to an open corresponding pin on a device connector, the sensing signal of that signal pin sees some impedance due to a load associated with one or more sensor of the intraluminal device connected to the device connector. In that case, the plurality of signals carried by the plurality of pins of the PIM connector experiences a change of impedance that can be detected by the PIM and be used by the PIM to identify the sensor type of the currently connected intraluminal device. In some embodiments, the change of impedance in the plurality of signal can wake up the PIM from the “sleeping mode.” The same applies to the power pins of the PIM connector. When any of the power pins is not connected to an open corresponding pin on a device connector, the standby voltage of that power pin results in a current output to one or more sensor of the intraluminal device connected to the device connector. In that case, the plurality of signals carried by the plurality of pins of the PIM connector experiences a change of current output that can be detected by the PIM and be used by the PIM to identify the sensor type of the currently connected intraluminal device. In some embodiments, the change of current output in the plurality of signal can wake up the PIM from the “sleeping mode.”

After the PIM, such as the PIM 202 in FIG. 2, identifies the sensor type of the intraluminal device currently connected to itself via the connector pair (i.e. a PIM connector and a compatible device connector), the PIM would configure itself and/or a console, such the console 201 in FIG. 2, based on attributes of the identified sensor type. In some embodiments, the PIM can include multiple components, such as signal amplifiers, signal filters, physical layer signal modulators, and analog to digital converter (ADC). These components require different parameters to correctly operate with different type of sensors. Configuring the PIM involves at least changing or resetting these parameters so that the PIM can function properly with the connected intraluminal device. In some implementations, the console also needs to be configured by the PIM once the sensor type of the intraluminal device is identified, such that the console can properly process the medical data obtained by the intraluminal device and generate a graphical representation of the medical data.

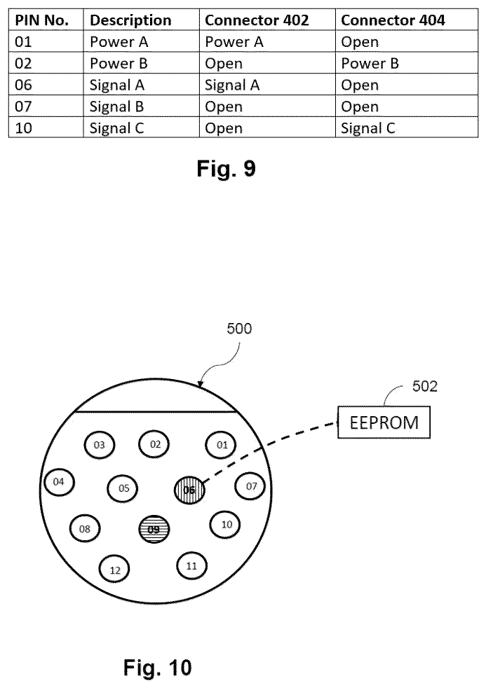

Referring now to FIG. 10, shown therein is a schematic diagram of a device connector 500 configured to engage a PIM connector, such as the PIM connector 204. In some embodiments, the device connector 500 includes a pin that is electrically connected to a memory, such as an electrically erasable programmable read-only memory (EEPROM) 502 (or EEPROM 502). The EEPROM 502 is pre-programmed by an EEPROM programmer or writer such that the EEPROM 502 stores information or data that can be used to uniquely identify the intraluminal device connected to the device connector 500 and the type of the sensor installed on the intraluminal device. For the case of reference, such information or data may be referred to as the device identifier data. In the example shown in FIG. 10, the EEPROM 502 is electrically connected to pin 06 corresponding to the pin 06 of the PIM connector. The pin 06 of the PIM connector is electrically connected to an EEPROM reader disposed within the PIM. The EEPROM reader in the PIM can read the device identifier data off of pin 06 and uniquely identify the intraluminal device and the type of sensors. Similar to what is described above, once the intraluminal device is identified by the PIM, the PIM can configure itself and/or the console according to the attributes of the intraluminal device so that the PIM and/or the console can function properly with the intraluminal device. As the EEPROM reader in the PIM reads the device identifier data stored in the EEPROM, the signals carried by the plurality of pins of the PIM connector can be said to experience a change.

For case of reference, the embodiments directed to identification of intraluminal devices by detection of changes in impedance and current output, as described in conjunction with FIGS. 3A to 9, can be referred to as the pin subset embodiments. The pin subset embodiments can be used alone, or in connection with the EEPROM embodiments described in conjunction with FIG. 10. In some embodiments, the pin subset embodiments can be used as the primary means for identifying the sensor type of the intraluminal device and the EEPROM embodiments can be used as the secondary means for the same purpose. In some other embodiments, the EEPROM embodiments can be used as the primary means for identifying the sensor type of the intraluminal device, and the pin subset embodiments can be used as the secondary means for the same purposes. In some implementations, the secondary means serve to confirm the identification generated by the primary means. If the secondary means generates an inconsistent identification, the PIM, such as the PIM 202, can prompt the console to display an alert to the user. The user can request the PIM to identify the intraluminal device again or manually enters the identification to initiate the configuration of the PIM or the whole intraluminal medical system. In some implementations, the primary means identifies the intraluminal device and the secondary means identifies further attributes associated with the identified intraluminal device. That is, in these implementations, the secondary means serves to augment the identification generated by the primary means. These further attributes identified by the secondary means can be used by the PIM to more thoroughly or precisely configure the PIM or the whole intraluminal medical system.

Persons skilled in the art will recognize that the apparatus, systems, and methods described above can be modified in various ways. While in the present disclosure it is referred primarily to intraluminal medical devices in general and intraluminal ultrasound devices in exemplary embodiments, in alternative embodiments at least one of the intraluminal medical device is an intraluminal sensing device configured to provide physiological measurements (e.g. pressure, flow velocity) within the lumen of the body. Additionally or alternatively, in an embodiment the medical devices may comprise at least one extracorporeal imaging device (e.g. ultrasound) and/or extracorporeal sensing device (e.g. electrocardiogram) besides intraluminal medical devices. The alternative embodiments have the same benefits as already mentioned in the detailed description of the intraluminal medical system 200. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A medical system, comprising:
a patient interface module (PIM) configured to selectively communicate with a first device or a second device different than the first device,
wherein the first device comprises a first sensor type configured to obtain first medical data associated with a body of a patient,
wherein the second device comprises a different, second sensor type configured to obtain second medical data associated with the body,
wherein the PIM comprises a PIM connector,
wherein the first device comprises a first connector configured to engage the PIM connector and the second device comprises a second connector configured to engage the PIM connector,
wherein a pin configured to carry the power in the first connector comprises a first voltage such that the first voltage comprises the power for operation of the first sensor type to obtain the first medical data,
wherein the pin configured to carry the power in the second connector comprises a second voltage such that the second voltage comprises the power for operation of the second sensor type to obtain the second medical data,
wherein the PIM is configured to identify the first sensor type and the second sensor type based on the first voltage and the second voltage such that the identification is performed using the power for the operation of the first sensor type and the operation of the second sensor type.

2. The system of claim 1,
wherein the first and second devices are intraluminal devices,
wherein the first medical data and the second medical data are associated with a body lumen of the patient while positioned within the body lumen.

3. The system of claim 1, further comprising the first device and the second device.

4. The system of claim 3, wherein the first sensor type comprises a first type of ultrasound transducers and the second sensor type comprises a second type of ultrasound transducers, the first type of ultrasound transducers different from the second type of ultrasound transducers.

5. The system of claim 3,
wherein the first sensor type comprises ultrasound transducers with a first center frequency, and
wherein the second sensor type comprises ultrasound transducers with a second center frequency different from the first center frequency.

6. The system of claim 1, wherein the first connector, the second connector, and the PIM connector comprise a same quantity of pins.

7. The system of claim 1, wherein the PIM is configured to:
identify the first sensor type when the first connector engages the PIM connector; and
identify the second sensor type when the second connector engages the PIM connector.

8. The system of claim 1,
wherein the PIM is configured based on the first sensor type when the PIM connector engages the first connector of the first device, and
wherein the PIM is configured based on the second sensor type when the PIM connector engages the second connector of the second device.

9. The system of claim 1, wherein the PIM is in communication with a console.

10. The system of claim 1,
wherein the console is configured based on the first sensor type when the PIM connector engages the first connector of the first device, and
wherein the console is configured based on the second sensor type when the PIM connector engages the second connector of the second device.

11. The system of claim 1,
wherein the first connector and the second connector each comprises a first pin and a second pin arranged in a physical layout,
wherein the first pin comprises a first physical location in the physical layout and the second pin comprises a second physical location in the physical layout,
wherein the pin configured to carry power in the first connector is the first pin and the pin configured to carry the power in the second connector is the second pin such that there is a difference between the first connector and the second connector in where the pin configured to carry the power is physically located.

12. The system of claim 11, wherein the physical layout of the first connector and the physical layout of the second connector are the same.

13. The system of claim 11, wherein the physical layout of the first connector and the physical layout of the second connector are different.

14. The system of claim 11, wherein the PIM is further configured to identify the first sensor type and the second sensor type based on the difference between the first connector and the second connector in where the pin configured to carry the power is physically located.

15. The system of claim 11,
wherein the PIM connector comprises the first pin and the second pin arranged in the physical layout,
wherein the first pin in the PIM connector corresponds to the first pin in the first connector and the second pin in the PIM connector corresponds to the second pin in the second connector.

16. The system of claim 11,
wherein the first connector and the second connector each comprises a third pin and a fourth pin arranged in the physical layout,
wherein the third pin comprises a third physical location in the physical layout and the fourth pin comprises a fourth physical location in the physical layout,
wherein a pin configured to provide electrical ground in the first connector is the third pin and the pin configured to provide the electrical ground in the second connector is the fourth pin such that there is a difference between the first connector and the second connector in where the pin configured to provide the electrical ground is physically located.

17. The system of claim 16,
wherein the first connector and the second connector each comprises a fifth pin and a sixth pin arranged in the physical layout,
wherein the fifth pin comprises a fifth physical location in the physical layout and the sixth pin comprises a sixth physical location in the physical layout,
wherein a pin configured to carry at least one of control signals or medical data in the first connector is the fifth pin and the pin configured to carry at least one of the control signals or the medical data in the sixth connector is the sixth pin such that there is a difference between the first connector and the second connector in where the pin configured to carry at least one of the control signals or the medical data is physically located.
